(19) United States
(12) Patent Application Publication
(10) Pub. No.: US 2018/0338778 A1
(43) Pub. Date: Nov. 29, 2018

(54) TROCAR

(71) Applicant: Surgical Innovations Limited, Leeds, West Yorkshire (GB)

(72) Inventors: David Andrew Main, Leeds, West Yorkshire (GB); Christopher David Harrison, Leeds, West Yorkshire (GB)

(73) Assignee: Surgical Innovations Limited, Leeds, West Yorkshire (GB)

(21) Appl. No.: 15/777,873

(22) PCT Filed: Dec. 22, 2016
(86) PCT No.: PCT/GB2016/000219

§ 371 Date: May 21, 2018

(30) Foreign Application Priority Data
Dec. 23, 2015 (GB) 1522719.2

(51) Int. Cl. A61B 17/34

(52) U.S. Cl. CPC A61B 17/3423; A61B 2017/00477; A61B 17/3462

(57) ABSTRACT

An obturator includes an elongate shaft extending between a proximal end and a distal end, the elongate shaft having an opening at the proximal end and defining an elongate cavity which is sized and shaped to receive an optical instrument. The obturator is provided with an axially extending finger for radial movement inwardly to engage the outer surface of the optical instrument located within the elongate cavity. The finger may be a latch member with a latch element and able to be moved between a first position, in which the latch element is located relatively radially inward, and a second position in which the latch element is located relatively radially outward. With the obturator located within a cannula body, an instrument inserted into the obturator causes the latch element of the latch member to engage with the cannula to prevent relative axial movement therebetween.

### FIELD OF THE INVENTION

The invention relates to trocar systems including obturators and, more particularly, to obturators having hollow shafts for the insertion of instruments, including, but not limited to, optical instruments.

### BACKGROUND TO THE INVENTION

A trocar obturator is used to separate body tissue during insertion through a body wall. The obturator comprises an elongate shaft which extends along a longitudinal axis between a proximal end and a distal end. The distal tip of an optical obturator is formed from a transparent material to enable visualisation of tissue during the insertion of the obturator through the body wall. The obturator is shaped and dimensioned to enable the insertion of a conventional laparoscope which typically includes an imaging element and fibre optic light fibres. During use, the obturator is first inserted into and through a trocar seal and cannula. A conventional laparoscope is then inserted into the proximal end of the blade less obturator and advanced to the distal tip of the obturator.

An endoscopic video camera is attached to the proximal end of the laparoscope and the trocar system is then axially advanced by the surgeon through the body wall. The surgeon can visually observe the tissue as it is being separated via a video monitor that is connected to the endoscopic video camera.

### STATEMENTS OF THE INVENTION

According to a first aspect of the present invention, there is provided an obturator comprising an elongate shaft extending between a proximal end and a distal end, the elongate shaft having an opening at the proximal end and defining an elongate cavity which is sized and shaped to receive an optical instrument, the obturator being provided with an axially extending finger which is arranged for radial movement inwardly so as to engage the outer surface of said optical instrument located within said cavity.

Preferably, two or more such fingers are provided and, more preferably, four fingers are provided.

Preferably, the or each finger is integral with said elongate shaft.

According to a second aspect of the present invention, there is provided an obturator comprising an elongate shaft extending between a proximal end and a distal end, the elongate shaft having an opening at the proximal end and defining an elongate cavity which is sized and shaped to receive an optical instrument, the obturator being provided with a latch member provided with a latch element and movable between a first position in which the latch element is located relatively radially inward and a second position in which the latch element is located relatively radially outward, whereby, with the obturator located within a cannula body, an instrument inserted into the obturator causes the latch element of the latch member to engage with the cannula to prevent relative axial movement therebetween.

Preferably, there are two or more latch members. Preferably, the or each latch member is integral with said elongate shaft.

The latch member may have an outwardly extending leg which may be pushed inwardly to release the latch element from engagement with the cannula.

Alternatively, separate release means are provided to enable disengagement of the latch element from the cannula.

The invention also provides an obturator with one or more fingers as referred to above together with one or more latch members. For instance, two fingers and two latch members may be provided with the latch members peripherally interposed between fingers.

The invention further provides a surgical access device comprising a cannula and an obturator of the invention.

### BRIEF DESCRIPTION OF THE DRAWINGS

The accompanying drawings are as follows:

FIG. 1 is a perspective view of a first embodiment of an obturator in accordance with the present invention;

FIG. 2 is a longitudinal section of the obturator of FIG. 1 with the fingers in their relaxed (unflexed) positions;

FIG. 3 is a longitudinal section of the obturator of FIG. 1 with the fingers flexed inwardly;

FIG. 4 is a longitudinal section of the obturator of FIG. 1 showing the obturator during insertion into a cannula;

FIG. 5 is a longitudinal section of the obturator of FIG. 1 showing the obturator fully inserted into the cannula;

FIG. 6 is a longitudinal section of the obturator of FIG. 1 showing the obturator fully inserted into the cannula and an optical instrument entering into the obturator;

FIG. 7 is a longitudinal section of a second embodiment of an obturator of the invention showing the obturator inserted into a cannula and with the latch member in its unlatched position; and

FIG. 8 is a longitudinal section of the obturator of FIG. 7 showing the obturator inserted into a cannula and with the latch member in its latching position.

### DETAILED DESCRIPTION OF THE INVENTION

Embodiments of the present invention will now be described, by way of examples only, with reference to the accompanying drawings.

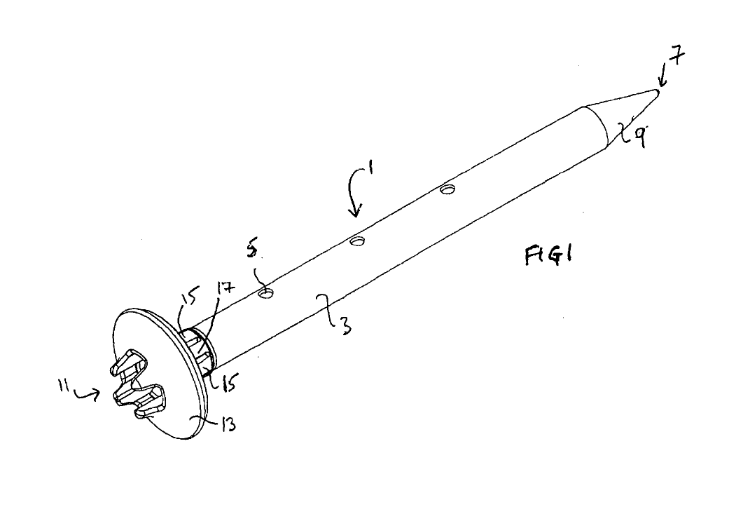

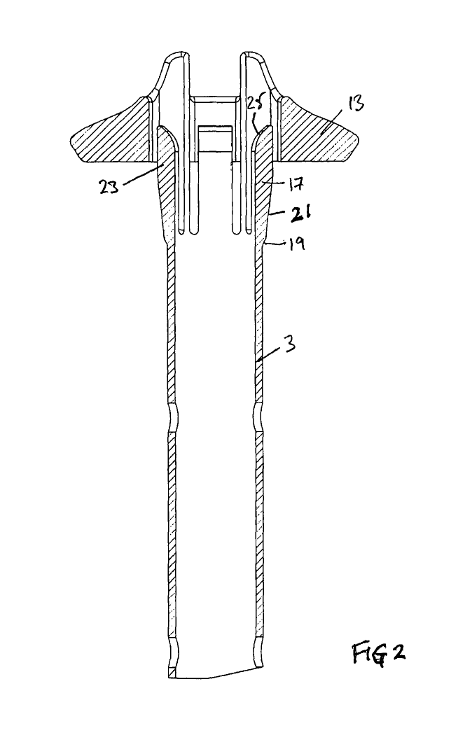

Referring to FIGS. 1 and 2 of the accompanying drawings, an obturator 1 comprises an elongate shaft 3 having, at its distal end 7, a transparent tip 9.

At its proximal end 11, obturator 1 is provided with an integral shaped cap 13 which is linked to the shaft 3 by four longitudinal arms 15 which are equally spaced apart in a peripheral sense. The linkage between the body of shaft 3 and the cap 13 is a rigid one and the outer surface of each arm 15 is at the same radial distance from the shaft axis as is the shaft body.

Located between each pair of arms 15 is an integral finger 17 which extends from the body of shaft 3, to which it is attached, longitudinally in a proximal direction and terminating at free end 19 within cap 13. The connection between the finger 17 and the body of shaft 3 is such that the finger 17 can flex radially inwardly although with limited movement in a radially outward direction due to the proximity of cap 13.

Although the inner surface of each finger 17 has a diameter which is the same as that of the body of shaft 3, the outer surface is stepped outwardly at its distal end 19, is inclined outwardly over a portion 21 and has a constant diameter over a portion 23 towards its proximal end. The proximal 25 end of finger 17 curves upwardly and outwardly from its inner to its outer surface.

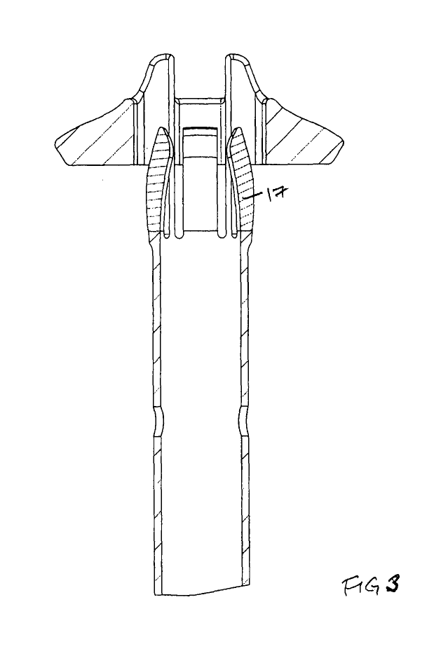

Referring to FIG. 3 of the accompanying drawings, this figure illustrates the position of fingers 17 when they are pushed radially inwardly. It can be seen that the inner surface of each finger 17 then lies radially inwards of the inner surface of the body of shaft 3.

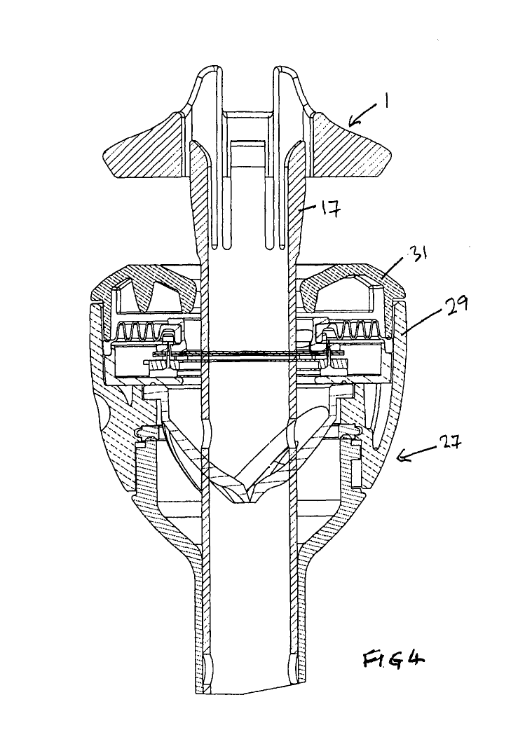

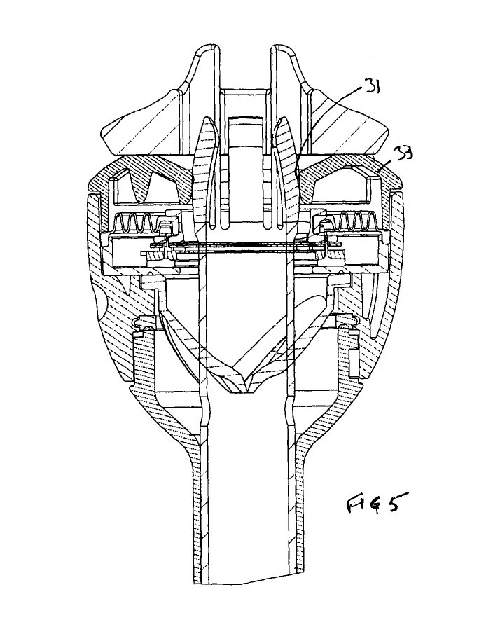

Referring to FIGS. 4 and 5 of the accompanying drawings, there is illustrated the position of fingers 17 as the obturator 1 is pushed into cannula 27, the Figures showing the seal 29 at the proximal end of the cannula. FIG. 4 shows the position prior to entry of the finger bearing portion of the obturator 1 into the seal. It can be seen that the fingers 17 are in their relaxed (unflexed) positions. As the obturator 1 is further pushed into cannula 27, fingers 17 come into engagement with inner surface 31 of end cap 33 of seal 29. This causes fingers 17 to flex inwardly to the position shown in FIG. 5 where the inner surface of fingers 17 now extend a short distance radially inwards of the inner surface of shaft 3.

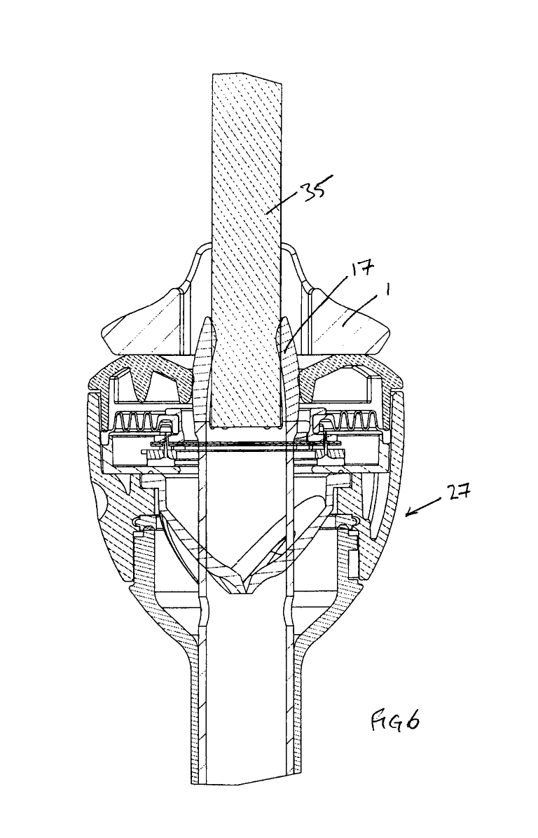

Referring to FIG. 6 of the accompanying drawings, there is illustrated the introduction into obturator 1, located within cannula 27 of an optical instrument 35. It can be seen that fingers 17 are in gripping engagement with the outer surface of instrument 35, thereby restraining or inhibiting the axial movement of the instrument 35 relative to the obturator 1.

Once the instrument 35 has been removed from obturator 1 and the latter is removed from cannula 27, the fingers return to their original (unflexed) positions.

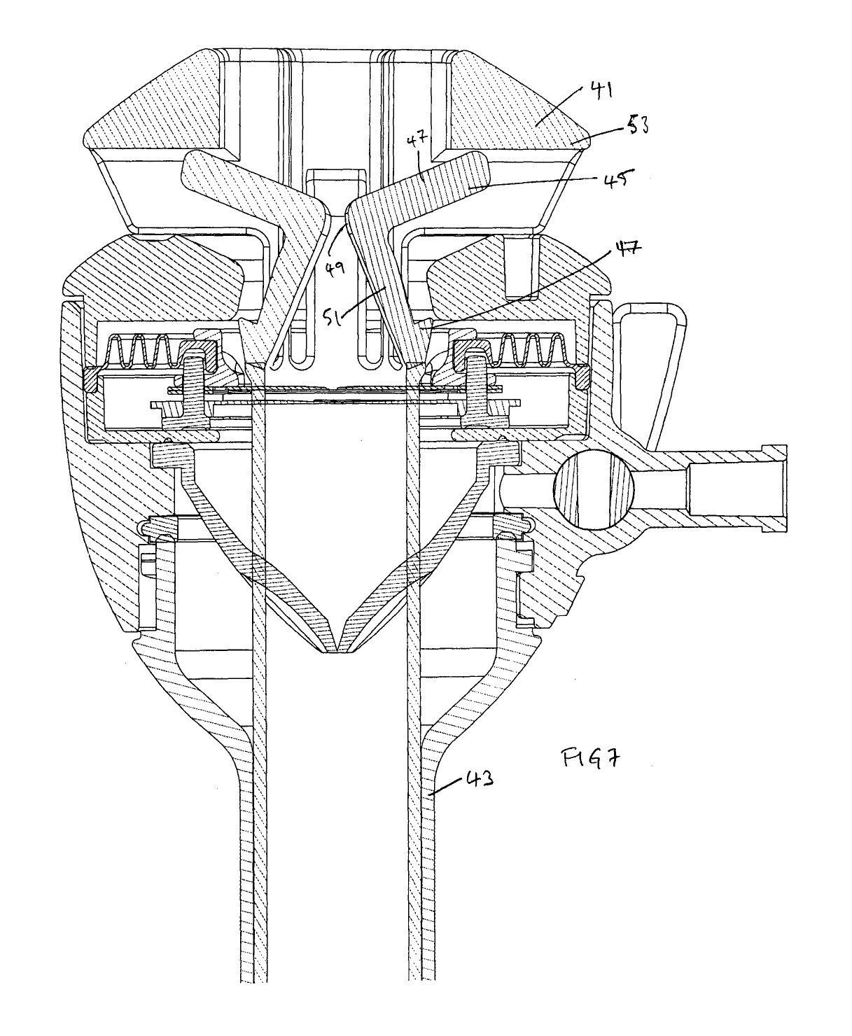

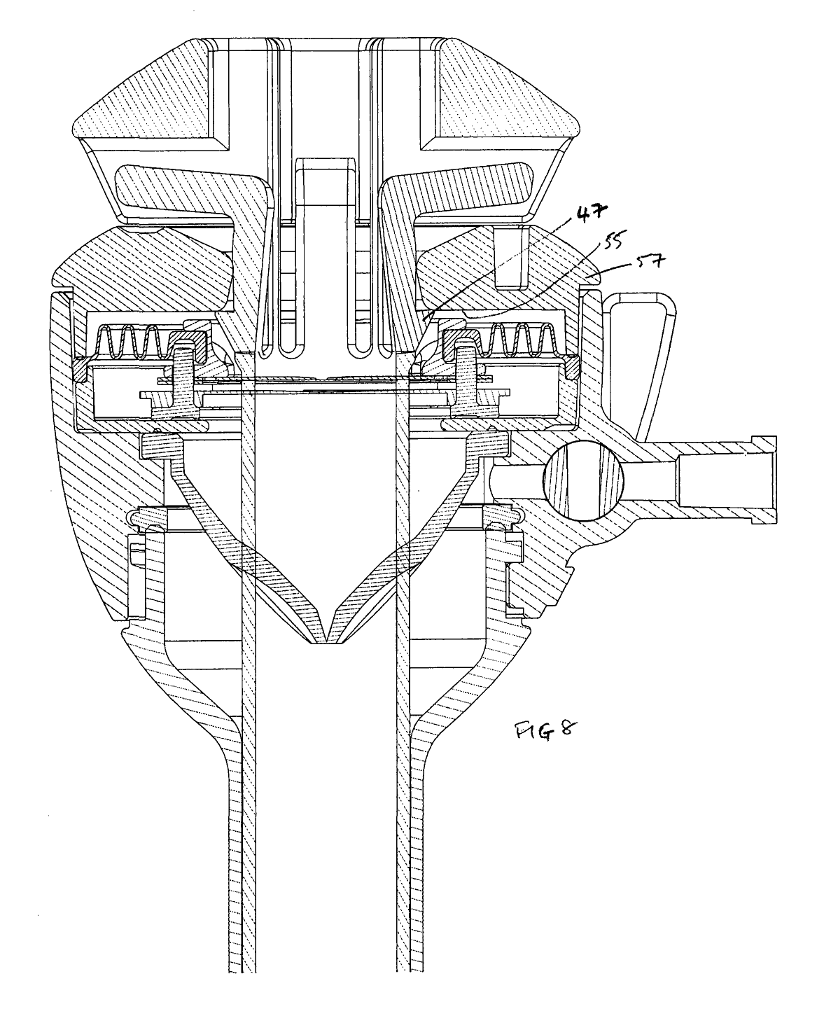

Referring to FIGS. 7 and 8 of the accompanying drawings, a second embodiment of the present invention is an obturator 41 which is shown located within a cannula 43. The obturator is similar to that described above with reference to FIGS. 1 to 6 except that two of the fingers 17, which are oppositely disposed, are substantially L-shaped latch members 45 each having legs 47 and 51. Lower leg 51 connects to, and is integral with, shaft 50 and upper leg 47 extends from the upper end of leg 51 into a radial channel 48. Lower leg 51 of latch member 45 is provided at its free end with a latch element in the form of barb 52 which extends radially outwards.

Insertion of an optical instrument into obturator 41 causes latch members 45 to move from the positions shown in FIG. 7 to those in FIG. 8 where the barbs 52 engage with the under surface 55 of cannula cap 57. Accordingly, the obturator 1 is now latched into the cannula body.

In order to release and remove the obturator 1, the radially outward ends 59 of latch members 59 are pushed inwardly to release the barbs 52 from their engagement with surface 55 of cannula cap 57.

In an alternative embodiment, one or more separate release buttons may be provided to unlatch the latch members and allow removal of the optical instrument from the obturator.

In another alternative embodiment there are no fingers 17 and only latching members are provided.

Although the above embodiments have been described for use with optical instruments, it will be appreciated that they could be used with other types of instrument.

1. An obturator comprising an elongate shaft extending between a proximal end and a distal end, the elongate shaft having an opening at the proximal end and defining an elongate cavity is sized and shaped to receive an optical instrument, the obturator having an axially extending finger for radial movement inwardly for engaging the outer surface of said optical instrument located within said cavity.

2. An obturator according to claim 1, wherein each finger is integral with said elongate shaft.

3. An obturator comprising an elongate shaft extending between a proximal end and a distal end, the elongate shaft having an opening at the proximal end and defining an elongate cavity sized and shaped to receive an optical instrument, the obturator having a latch member with a latch element and movable between a first position, in which the latch element is located relatively radially inward, and a second position in which the latch element is located relatively radially outward, whereby, with the obturator located within a cannula body, an instrument inserted into the obturator causes the latch element of the latch member to engage with the cannula to prevent relative axial movement therebetween.

4. An obturator according to claim 3, wherein the latch member is integral with said elongate shaft.

5. An obturator according to claim 3, wherein the latch member has an outwardly extending leg which are able to be pushed inwardly to release the latch element from engagement with the cannula.

6. An obturator according to claim 3, further comprising release means for enabling disengagement of the latch element from the cannula.

7. An obturator according to claim 1, wherein the obturator includes a latch member with a latch element and movable between a first position, in which the latch element is located relatively radially inward, and a second position in which the latch element is located relatively radially outward, whereby, with the obturator located within a cannula body, an instrument inserted into the obturator causes the latch element of the latch member to engage with the cannula to prevent relative axial movement therebetween.

8. An obturator according to claim 7, wherein the latch member is integral with said elongate shaft.

9. An obturator according to claim 7, wherein the latch member has an outwardly extending leg which is able to be pushed inwardly to release the latch element from engagement with the cannula.

10. An obturator according to claim 7, a further comprising separate release means for enabling disengagement of the latch element from the cannula.

11-12. (canceled)

13. A surgical access device, comprising:
a cannula having a cannula body; and,
an obturator including an elongate shaft extending between a proximal end and a distal end, the elongate shaft having an opening at the proximal end and defining an elongate cavity sized and shaped to receive an optical instrument, the obturator having an axially extending finger for radial movement inwardly for engaging the outer surface of said optical instrument located within said cavity, said obturator being located within said cannula body.

14. A surgical access device according to claim 13, wherein each finger is integral with said elongate shaft.

15. A surgical access device according to claim 13, wherein the obturator includes a latch member with a latch element and movable between a first position, in which the latch element is located relatively radially inward, and a second position in which the latch element is located relatively radially outward, whereby, with the obturator located within a cannula body, an instrument inserted into the obturator causes the latch element of the latch member to engage with the cannula to prevent relative axial movement therebetween.

16. A surgical access device according to claim 15, wherein the latch member is integral with said elongate shaft.

17. A surgical access device according to claim 15, wherein the latch member has an outwardly extending leg which is able to be pushed inwardly to release the latch element from engagement with said cannula.

18. A surgical access device according to claim 15, further comprising separate release means for enabling disengagement of the latch element from the cannula.
